(19) United States
(12) Patent Application Publication
(10) Pub. No.: US 2015/0307774 A1
(43) Pub. Date: Oct. 29, 2015

(54) Chelates, chelating agents, conjugates derived thereof and their use

(71) Applicant: Qi WANG, Turku (FI)

(72) Inventor: Qi Wang, Turku (FI)

(21) Appl. No.: 14/430,570

(22) PCT Filed: Sep. 20, 2013
(86) PCT No.: PCT/FI2013/050912

§ 371 Date: Mar. 24, 2015

(30) Foreign Application Priority Data
Sep. 24, 2012 (FI) 20120311

(51) Int. Cl. C09K 11/06; C12N 15/11; G01N 33/533; C07K 2/00

(52) U.S. Cl. CPC C09K 11/06; C07K 2/00; C12N 15/11; G01N 33/533

(57) ABSTRACT

This invention relates to a group of novel stable and luminescent lanthanide(III) chelates and chelating agents. This invention further relates to a detectable molecule comprising the lanthanide chelate and the use of the molecule in a method of carrying out a biospecific binding assay.

Suggested figure:

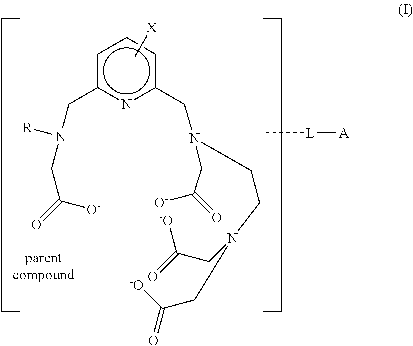

### FIELD

This invention relates to stable luminescent lanthanide(III) chelates and biomolecules labeled with these chelates. This invention further relates to the use of the chelates in a method of carrying out a biospecific binding assay. The invention relates also to chelating agents useful in solid phase synthesis of oligopeptides and oligonucleotides and oligopeptide and oligonucleotide conjugates so obtained.

### BACKGROUND

Luminescent lanthanide(III) chelates have several special properties that make them excellent tools in especially homogenous bioaffinity assays. Their large Stokes' shift has a decreasing effect on scattering phenomena. The long fluorescence decay after excitation of these molecules allow time-resolved signal detection, which eliminates completely the background luminescence originating, e.g. from buffer components, plastics, and biomaterials. The very narrow emission lines allow the use of effective filters which diminish the background. Furthermore, since the lanthanide(III) chelates do not suffer from concentration quenching it is possible to have several chelates in close proximity enabling multilabeling. This phenomenom allows also the development of chelates bearing several light absorbing moieties.

However, the use of stable chelates in bioaffinity assays demands optimization of the chelate structure. The optimal luminescent lanthanide chelate must be stable in the presence of additional chelators even at low pH and high temperature. The chelate should have optimal emission profile, high hydrophilicity, small size, good biocompatibility, little effect on biomolecules and good energy transfer properties. In addition, the chelate should be feasible for different energy acceptors, and when possible, the synthesis of the molecule should be simple, cheap and scalable.

Europium(III) chelate of 4-[2—(4-isothiocyanatophenyl)ethynyl]-2,6,-bis{[N,N-bis(carboxymethyl)-amino]methyl}pyridine [U.S. Pat. No. 4,920,195]is one of the most commonly used biomolecule labeling reagent, since it fulfills almost all of the above mentioned requirement. The most serious drawback of this chelate as well as other 7-dentate lanthanide(III) chelates is that it exhibits rather low chelate stability limiting its use in applications involving treatments at elevated temperature, low pH and in the presence of additional chelating agents such as EDTA.

The chelate stability can be increased by using cyclic chelating moieties [U.S. Pat. No. 4,920,195; WO2005058877; WO2005021538]but neutralization of the net charge decreases the water solubility of the chelate. The decreased solubility of the cyclic chelates can be enhanced by using hydrophilic groups at the aromatic unit [WO2009030819]. The chelate stability can be enhanced also by increasing the number of chelating carboxylic acids [WO2008020113, US2004166585]. However, all the prior art methods for increasing chelate stability of chelates including pyridine subunit also increase the size of the chelate and/or decrease the chelate solubility.

### SUMMARY

An object of the present technology is to alleviate and eliminate the problems related to the luminescent lanthanide chelates of prior art.

According to one aspect this technology concerns chelates including a lanthanide(III) ion selected from europium, terbium, samarium and dysprosium and a chelating ligand of formula (I)

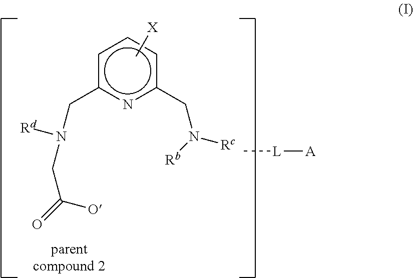

wherein

X is an aromatic unit,

L is a linker formed from one to ten moieties, each moiety being selected from the group consisting of phenylene, alkylene containing 1-12 carbon atoms, ethynydiyl (—C═C—), ethylenediyl (—C═C—), ether (—O—), thioether (—S—), amide (—CO—NH—, —CO—NR′—, —NH—CO— and —NR′—CO—), carbonyl (—CO—), ester (—COO— and —OOC—), disulfide (—SS—), sulfonamide (—SO₂—NH—, —SO₂—NR′—), sulfone (—SO₂—), phosphate (—O—PO₂—O—), diaza (—N═N—), and tertiary amine, wherein R′ represents an alkyl group containing less than 5 carbon atoms, and L replacing a hydrogen anywhere in the parent compound, or not present,

A is a reactive group selected from isothiocyanate, bromoacetamido, iodoacetamido, maleimido, 4,6-dichloro-1,3,5-triazin-2-ylamino, pyridyldithio, thioester, aminooxy, azide, hydrazide, amino, alkyne, a methacroyl group, carboxylic acid, acid halide, and an active ester,

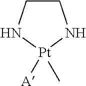

wherein A′ is cleaving group selected from Cl, (CH₃)₂SO, H₂O, and NO₃ wherein—is the position of the linker L and

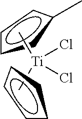

wherein—is the position of linker L,

and A replacing a hydrogen of the aromatic unit X when the linker L is not present, and

R^{a} is selected from —CH₂COO⁻ and —(CH₂)ₙN(CH₂COO⁻)₂, wherein n is 2 or 3, and

R^{b} is —(CH₂)ₘN(CH₂COO⁻)₂, wherein m is 2 or 3, and

R^{c} is selected from CH₂COO—, and —(CH₂)ₜN(CH₂COO⁻)₂, wherein 1 is 2 or 3.

According to another aspect this technology concerns a chelating agent of formula II

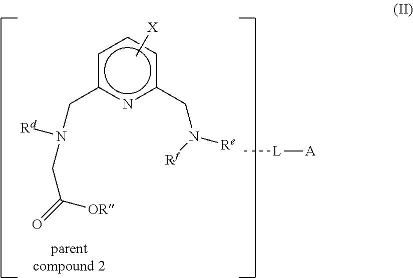

wherein X is an aromatic unit,

L is a linker formed from one to ten moieties, each moiety being selected from the group consisting of phenylene, alkylene containing 1-12 carbon atoms, ethynydiyl (—C≡C—), ethylenediyl (—C═C—), ether (—O—), thioether (—S—), amide (—CO—NH—, —CO—NR′—, —NH—CO— and —NR′—CO—), carbonyl (—CO—), ester (—COO— and —OOC—), disulfide (—SS—), sulfonamide (—SO₂—NH—, —SO₂—NR′—), sulfone (—SO₂—), phosphate (—O—PO₂—O—), diaza (—N═N—), and tertiary amine, wherein R′ represents an alkyl group containing less than 5 carbon atoms and replacing a hydrogen anywhere in the parent compound 2,

A is a reactive group selected from the group consisting of carboxylic acid or its salt, acid halide, carboxylic acid ester, an amino acid residue —CH(NHR¹)R² where R¹ is a transient protecting group and R²is a carboxylic acid or its salt, carboxylic acid halide or an active ester and a group of —Z¹—O—PZ²—0—R³ where one or two of the oxygen atoms optionally is replaced by sulfur, Z² is chloro or NR⁴R⁵, R³ is a protecting group, R⁴ and R⁵ are alkyl groups including 1-8 carbons, Z¹ is absent or is a radical of a purine base or a pyrimidine base wherein the base is connected to the oxygen atom via either a) a hydrocarbon chain, which is substituted with a protected hydroxymethyl group, or b) a furan ring or pyrane ring, and

R^{d} is selected from —CH₂COOR″ and —(CH₂)ₙN(CH₂COOR″)₂, wherein n is 2 or 3,

R^{e} is —(CH₂)ₘN(CH₂COOR″)₂, whereinm is 2 or 3, and

R^{f} is selected from CH₂COOR″, and —(CH₂)1N(CH₂COOR″)₂, wherein 1 is 2 or 3 and wherein R″ is a protecting group.

According to another aspect this technology concerns a detectable molecule such as a biomolecule conjugated with a chelate according the present technology wherein the biomolecule is selected from the group consisting of oligopeptide, oligonucleotide, DNA, RNA, modified oligo- or polynucleotide, protein, oligosaccharide, polysaccharide, phospholipide, PNA, LNA, antibody, antigen, steroid, biotin, hapten, drug, receptor bindig ligand, and lectine.

According to another aspect this technology concerns a detectable molecule such as a biomolecule obtained by synthesis on a solid phase by introduction of a chelating agent according to the present technology into the biomolecule structure followed by deprotection and introduction of a lanthanide ion.

According to another aspect the present technology concerns a method of carrying out a specific bioaffinity assay using a biomolecule conjugate of the present technology with an analyte to be determined.

According to another aspect the present technology concerns a use of a biomolecule conjugate according to the present technology in a specific bioaffinity binding assay utilizing fluorometric or time-resolved fluorometric determination of a specific luminescence.

Further aspects of the present technology have been disclosed in dependent claims.

### BRIEF DESCRIPTION OF DRAWINGS

FIG. 1 discloses the structures of the chelate of prior art (1) and two representative chelates according to the present technology (6,7).

FIG. 2 discloses UV-Vis absorption, excitation (λₑₘ=615 nm) and emission (λ_{exc}=319 nm) spectra of 1b in 0.01 M TRIS/HCl buffer at pH 7.4.

FIG. 3 discloses emission spectra of the 1b, 6b and 7b complexes (from bottom to top) in TRIS/HCl buffer (0.01 M, pH 7.4; λ_{exc}=318 nm).

FIG. 4 discloses hCRP immunoassays with biotin-conjugated europium chelates: 1c (▪), 6c (), and 7c (▴). The lower limit of detections for the biotin chelates were 6.5, 1.5 and 1.9 μg/L, respectively.

### DETAILED DESCRIPTION

In this invention, it was observed that stability of a lanthanide chelate including a pyridine subunit can be enhanced without need to increase significantly the size of the chelating ligand. According to one embodiment, the present technology concerns chelates including a lanthanide(III) ion selected from europium, terbium, samarium and dysprosium and a chelating ligand of formula (I)

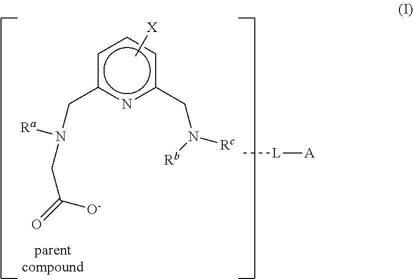

wherein

X is an aromatic unit,

L is a linker formed from one to ten moieties, each moiety being selected from the group consisting of phenylene, alkylene containing 1-12 carbon atoms, ethynydiyl (—C≡C—), ethylenediyl (—C═C—), ether (—O—), thioether (—S—), amide (—CO—NH—, —CO—NR′—, —NH—CO— and —NR′—CO—), carbonyl (—CO—), ester (—COO— and —OOC—), disulfide (—SS—), sulfonamide (—SO₂—NH—, —SO₂—NR′—), sulfone (—SO₂—), phosphate (—O—PO₂—O—), diaza (—N═N—), and tertiary amine, wherein R′ represents an alkyl group containing less than 5 carbon atoms, and L replacing a hydrogen anywhere in the parent compound or not present,

A is a reactive group selected from isothiocyanate, bromoacetamido, iodoacetamido, maleimido, 4,6-dichloro-1,3,5-triazin-2-ylamino, pyridyldithio, thioester, aminooxy, azide, hydrazide, amino, alkyne, a methacroyl group, carboxylic acid, acid halide, and an active ester,

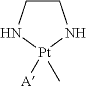

wherein A′ is cleaving group selected from Cl, (CH₃)₂SO, H₂O, and NO₃ wherein—is the position of the linker L and

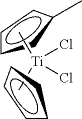

wherein—is the position of linker L,

and A replacing a hydrogen of the aromatic unit X when the linker L is not present, and

R^{a} is selected from —CH₂COO⁻ and —(CH₂)ₙN(CH₂COO⁻)₂, wherein n is 2 or 3, and

R^{b} is —(CH₂)mN(CH₂COO⁻)₂, wherein m is 2 or 3, and

R^{c} is selected from CH₂COO—, and —(CH₂)ₜN(CH₂COO⁻)₂, wherein m is 2 or 3.

According to a preferable embodiment R^{a} is selected from —CH₂COO⁻ and —CH₂CH₂N(CH₂COO⁻)₂, R^{b} is —(CH₂)₂N(CH₂COO⁻)₂, and R^{c} is CH₂COO⁻. According to this embodiment the chelates of the present technology have the following structures

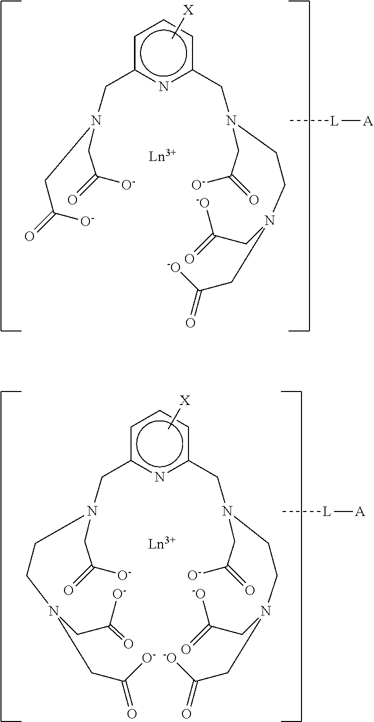

Wherein X, L and A are as defined above, and Ln is selected from Eu, Tb, Sm, and Dy.

As defined herein, the “parent compound” is the part of the chelating ligand of formula (I) that is between the square brackets. Accordingly, the parent compound is understood as

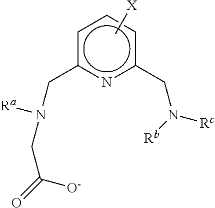

As defined herein, the “aromatic unit” is a chemical compound that contains conjugated planar ring system with delocalized pi electron cloud instead of discrete alternating single and double bonds. Exemplary aromatic units suitable for the present technology are phenylethynyl, furyl, thienyl, phenyl, and pyrrole groups, and their substituted derivatives. Exemplary substituents are alkyl groups and alkoxy groups. Methods to synthesize the pyridines with an aromatic group X are known in the art. Exemplary methods are disclosed in Bioconjugate Chemistry, 2009, vol 20, page 405, Scheme 1, incorporated here by reference.

As defined herein, “alkyl group” is linear or branched, like methyl, ethyl, n-propyl, i-propyl, n-butyl, t-butyl and sec-butyl group. The alkyl group can be tethered also to other groups like hydroxyl, carboxylic acid, carbohydrate and sulfonic acid groups.

As defined herein “alkoxy group” can be linear or branched, like methoxy, ethoxy, n-propoxy, i-propoxy, n-butoxyl, t-butoxyl and sec-butoxy group. The alkoxy group can be tethered also to other groups like hydroxyl, carbohydrate, carboxylic acid and sulfonic acid groups.

Exemplary alkoxy and alkyl substituted aromatic units are

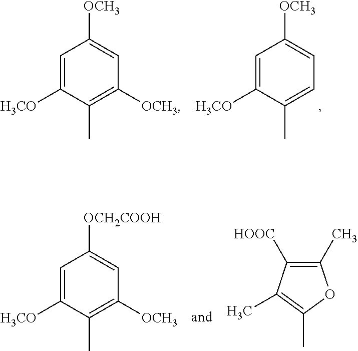

wherein—is the position of wherein the aromatic unit is connected to the pyridine unit and wherein —CH₂COOH and —COOH groups are exemplary reactive groups A.

The aromatic unit is connected to 3- or preferably to 4-position of the pyridine unit.

The chelate must bear a reactive group A in order to enable covalent binding to a detectable molecule such as to a biomolecule.

According to an embodiment, the reactive group A is selected from the group consisting isothiocyanate, bromoacetamido, iodoacetamido, maleimido, 4,6-dichloro-1,3,5-triazin-2-ylamino, pyridyldithio, thioester, aminooxy, azide, hydrazide, amino, alkyne, a polymerizable group such as methacroyl group, and a carboxylic acid or carboxylic acid halide or an active ester thereof. As defined herein “active ester” is an aryl ester, vinyl ester, or hydroxyamine ester. Exemplary active esters are nitrophenyl ester, pentafluorophenyl ester and N-hydroxysuccinimidyl ester.

It has been proposed [U.S. Pat. No. 5,985,566]that oligonucleotides, DNA, RNA, oligopeptides, proteins and lipids can be transformed statistically by using label molecules tethered to platinum derivatives. In nucleic acids these molecules react predominantly at N7 of guanine residues. When the chelates of the present technology are used for these labelling purposes an exemplary reactive group A is

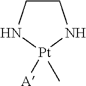

wherein A′ is cleaving group selected from Cl, (CH₃)₂SO, H₂O, and NO₃ wherein—is the position of the linker L.

When the chelate of the present technology is used for bioconjugation of phosphodiester linkages an exemplary reactive group A is

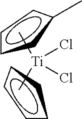

wherein—is the position of linker L.

In case the chelate should be attached to a microparticle or nanoparticle during the manufacturing process of said particles, the reactive group A is a polymerizable group, such as methacroyl group.

In the case the chelate is to be attached to solid supports including nanomaterials, biomolecules, and various organic molecules using copper(I) catalyzed Huisgen-Sharpless dipolar [2+3] cycloaddition reaction, the reactive group A has to be either azide or terminal alkyne.

The reactive group A can be attached to the parent compound either directly or via a linker L. When the reactive group is attached to the parent compound directly, the preferable position is the aromatic unit. Exemplary positions are 3- and 4-positions of phenylethynyl unit. According to a preferable embodiment the reactive group A is attached to 4-position of phenylethynyl group and it is selected from amino, isothiocyanate, iodoacetamido and 4,6-dichloro-1,3,5-triazin-2-ylamino groups. According to exemplary embodiments wherein the reactive groups are linked directly to the aromatic unit X are

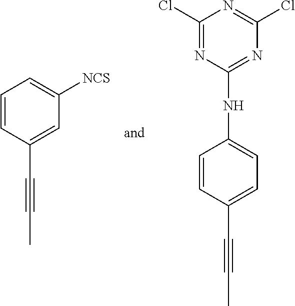

wherein—is the position of pyridine unit and the aromatic unit is phenylethynyl group.

According to one embodiment the reactive group is attached to the parent compound via a liker L. The linker is formed from one to ten moieties, each moiety being selected from the group consisting of phenylene, alkylene containing 1-12 carbon atoms, ethynydiyl (—C≡C—), ethylenediyl (—C═C—), ether (—O—), thioether (—S—), amide (—CO—NH—, —CO—NR′—, —NH—CO— and —NR′—CO—), carbonyl (—CO—), ester (—COO— and —OOC—), disulfide (—SS—), sulfonamide (—SO₂—NH—, —SO₂—NR′—), sulfone (—SO₂—), phosphate (—O—PO₂—O—), diaza (—N═N—), and tertiary amine, wherein R′ represents an alkyl group containing less than 5 carbon atoms and replacing a hydrogen anywhere in the parent compound. Exemplary positions are the pyridine unit, aromatic unit and the CH₂ units of the chelating part. The preferable position of the linker is the aromatic unit. The linker is preferable in applications wherein a space is needed between the chelate and the detectable molecule. An exemplary linker L reactive group A combination is

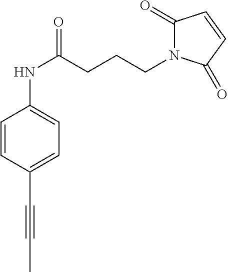 [SMILES: CC#Cc1ccc(NC(=O)CCCN2C(=O)C=CC2=O)cc1]

wherein—is the position of the pyridine unit.

Although organic chelators and their substituents have a significant effect on the photophysical properties of lanthanide(III) chelates, no general rules for the estimation of these effects are available. It has been proposed [U.S. Pat. No. 4,761,481]that electron releasing substituents in the aromatic moiety of phenyl and naphthyl substituted 2,6-[N,N-di(carboxyalkyl)aminoalkyl]pyridines have advantageous effects on the photophysical properties on their chelates with lanthanide ions. However, no experimental evidence was given. Later it has been shown that this is the case with various terbium(III) and dysprosium(III) chelates [U.S. patent application Ser. No. 11/004,061]but the corresponding europium(III) chelates are practically non-luminescent [Hemmilä et al., J. Biochem. Biophys. Methods, 1993, 26, 283]. Accordingly, it is clear for a person skilled in art that the choose of the lanthanide ion depends on the nature of the chromophore.

Exemplary chelates according to the present technology has the chelating ligand of formula (III)

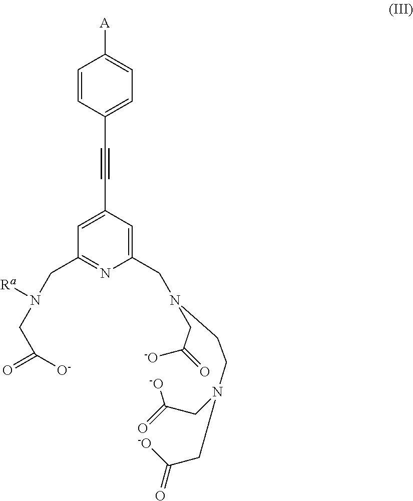

wherein the reactive group A is selected from amino, iodoacetamido, isothiocyanato and 4,6-dichloro-1,3,5-triazin-2-ylamino, and the lanthanide is selected from europium and samarium, preferably europium, and R^{a} is selected from —CH₂COO⁻ and —CH₂CH₂N(CH₂COO⁻)₂. According to an embodiment, R^{a} is CH₂COO⁻.

It is apparent that although the chelating carboxylic acid groups of the chelating ligands of formulas of this disclosure, such as formula (I) and formula (III), are drawn as deprotonated (i.e. —COO⁻), the formulas are only illustrative and the scope of the invention covers chelating ligands wherein one or more of the carboxylic acid groups are protonated (i.e. —COOH).

Exemplary chelates according to the present technology have the chelating ligand of formula (IV) and wherein the reactive group A is selected from amino, iodoacetamido, isothiocyanato and 4,6-dichloro-1,3,5-triazin-2-ylamino, and carboxyl group, and the lanthanide is selected from terbium and dysprosium. According to an embodiment the lanthanide is terbium.

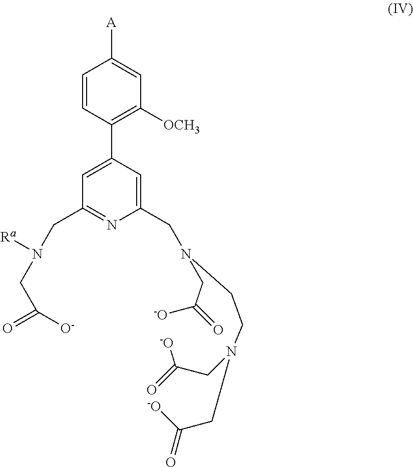

Exemplary chelates according to the present technology are

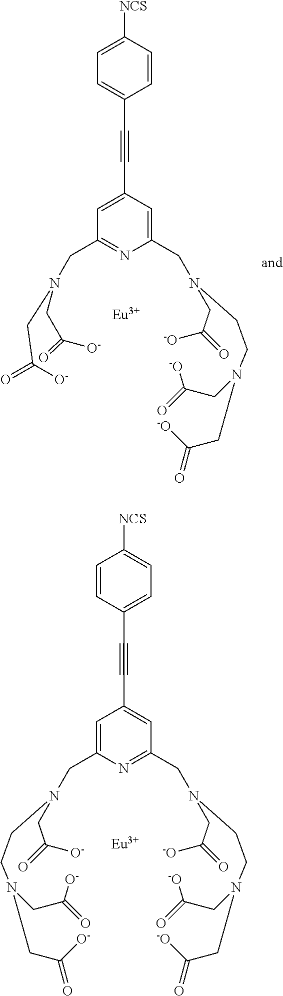

According to another embodiment the present technology concerns a detectable molecule such as a biomolecule conjugated with a chelate according to the present technology. The biomolecule is selected from the group consisting of oligopeptide, oligonucleotide, DNA, RNA, modified oligo- or polynucleotide, protein, oligosaccharide, polysaccharide, phospholipide, PNA, LNA, antibody, antigen steroid, biotin, hapten, drug, receptor bindig ligand, and lectine. The biomolecule can be labelled with the chelate of the present technology using methods known in the art. The position of labelling and the number of chelates conjugated can be chosen by reaction conditions employed ad by choosing the reactive group A according to the demands of the application and the detectable molecule to be labelled.

According to another embodiment this technology concerns a chelating agent of formula II

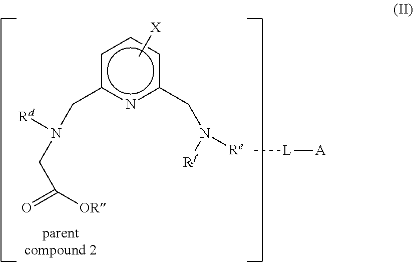

wherein X is an aromatic unit,

L is a linker formed from one to ten moieties, each moiety being selected from the group consisting of phenylene, alkylene containing 1-12 carbon atoms, ethynydiyl (—C≡C—), ethylenediyl (—C═C—), ether (—O—), thioether (—S—), amide (—CO—NH—, —CO—NR′—, —NH—CO— and —NR′—CO—), carbonyl (—CO—), ester (—COO— and —OOC—), disulfide (—SS—), sulfonamide (—SO₂—NH—, —SO₂—NR′—), sulfone (—SO₂—), phosphate (—O—PO₂—O—), diaza (—N═N—), and tertiary amine, wherein R′ represents an alkyl group containing less than 5 carbon atoms and replacing a hydrogen anywhere in the parent compound 2,

A is a reactive group selected from the group consisting of carboxylic acid or its salt, acid halide, carboxylic acid ester, an amino acid residue —CH(NHR¹)R² where R¹ is a transient protecting group and R² is a carboxylic acid or its salt, carboxylic acid halide or an active ester and a group of —Z¹—O—PZ²—O—R³ where one or two of the oxygen atoms optionally is replaced by sulfur, Z² is chloro or NR⁴R⁵, R³ is a protecting group, R⁴ and R⁵ are alkyl groups including 1-8 carbons, Z¹ is absent or is a radical of a purine base or a pyrimidine base wherein the base is connected to the oxygen atom via either a) a hydrocarbon chain, which is substituted with a protected hydroxymethyl group, or b) a furan ring or pyrane ring, and

R^{d} is selected from —CH₂COOR″ and —(CH₂)nN(CH₂COOR″)₂, wherein n is 2 or 3,

R^{b} is —(CH₂)ₘN(CH₂COOR″)₂, wherein m is 2 or 3, and

R^{c} is selected from CH₂COOR″, and —(CH₂)₁N(CH₂COOR″)₂, wherein 1 is 2 or 3 and wherein R″ is a protecting group.

According to a preferable embodiment R^{d} is selected from —CH₂COOR″ and —CH₂CH₂N(CH₂COOR″)₂, R^{b} is —(CH₂)₂N(CH₂COOR″)₂, and R^{c} is CH₂COOR″.

As defined herein, the “parent compound 2” is the part of the chelating ligand of formula (II) that is between the square brackets. Accordingly, the parent compound is understood as

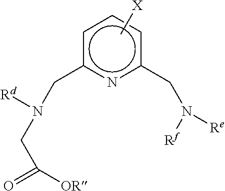

As defined herein, the “aromatic unit” is a chemical compound that contains conjugated planar ring system with delocalized pi electron cloud instead of discrete alternating single and double bonds. It is obvious for a person skilled in art that if the aromatic unit of chelating agent of formula (II) includes one of more functional groups such as amines, carboxylic acids, alcohols, or mercapto groups they must be protected to avoid harmful side reactions during solid phase chain assembly. It is also obvious that the protecting group has to be chosen according to the solid phase chemistry to be used.

The chelating agents according to the present technology may include different protecting groups. R″ is aimed to protect the chelating carboxylic acid groups during solid phase synthesis of biomolecules such as oligonucleotides and oligopeptides. R″ is chosen according to the synthesis strategy employed. Most commonly R″ is a permanent protecting group that is removed after completion of the chain assembly and before or during conversion of the biomolecule tethered to the chelating ligand to the corresponding lanthanide chelate. Exemplary protecting groups R″ applicable to oligonucleotide and oligopeptide chemistries are base and acid labile esters, respectively. The transient protecting groups are groups that are removed after each coupling step to allow chain elongation. Exemplary transient protecting groups for oligopeptide and oligonucleotide chemistries are base labile carbamates and highly acid labile ethers, respectively.

According to one embodiment, the chelating agent according to this invention is suitable for use in the synthesis of an oligopeptide. In this application, the reactive group A is connected to the chelating agent via a linker L, and A is a carboxylic acid or its salt, carboxylic acid halide or an ester or an amino acid residue —CH(NHR¹)R²where R¹ is a transient protecting group and R² is a carboxylic acid or its salt, carboxylic acid halide or an ester. A preferable halide is chloride.

In a preferable embodiment the transient protecting group R¹ is selected from a group consisting of Fmoc (fluorenylmethoxycarbonyl), Boc (tert-butyloxycarbonyl), or Bsmoc (1,1-dioxobenzo[b]thiophen-2-ylmethyloxycarbonyl), and R³ is a carboxylic acid or its salt, acid halide or an ester. In a preferable embodiment the protecting group R″ is tert-butyl that can be removed with TFA.

The chelating agent can be introduced into detectable molecules such as biomolecules with the aid of a peptide synthesizer as disclosed e.g. in EP 0967205. The chelating agent can be coupled to an amino tethered solid support or immobilized amino acid in the presence of an activator. When the condensation step is completed the transient amino protecting group of the chelating agent is selectively removed while the material is still attached to the solid support (e.g. with piperidine in the case of Fmoc-protecting group). Then, a second coupling of a chelating agent or other reagent (e.g. appropriately protected amino acid, steroid, hapten or organic molecule) is performed as above. When the synthesis of the desired molecule is completed, the material is detached from the solid support and deprotected. In Fmoc chemistry, the final cleavage and deptotection is performed by acid, such as TFA. Purification can be performed by HPLC techniques. Finally, the purified ligand is converted into the corresponding lanthanide(III) chelate by the addition of a known amount of lanthanide(III) ion.

Exemplary chelating agents of the present technology suitable for oligopeptide synthesis have the following structures:

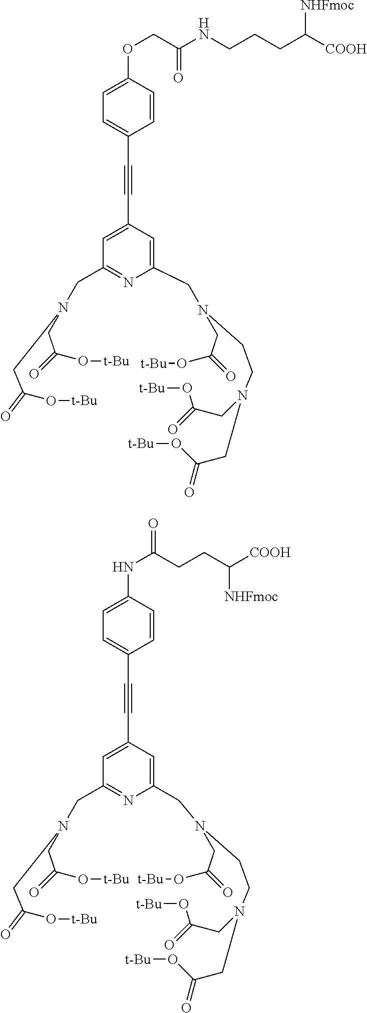

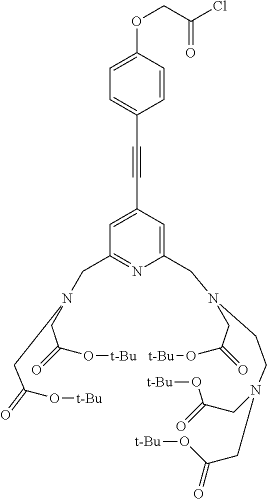 [SMILES: CC(C)(C)OC(=O)CN(CCN(CC(=O)OC(C)(C)C)Cc1cc(C#Cc2ccc(OCC(=O)Cl)cc2)cc(CN(CC(=O)OC(C)(C)C)CC(=O)OC(C)(C)C)n1)CC(=O)OC(C)(C)C]

According to another embodiment, the chelating agent according to the present technology is suitable for use in the synthesis of a labeled oligonucleotide on solid phase. In this case the reactive group A is connected to the chelating agent via a linker L, and A is —Z¹—O—PZ²—O—R³ wherein one of the oxygen atoms optionally is replaced by sulfur, Z² is chloro or NR⁴R⁵, R³ is a protecting group, R⁴ and R⁵ are alkyl groups comprising 1-8 carbons, and Z¹ is absent or is a radical of a purine base or a pyrimidine base or any other modified base suitable for use in the synthesis of modified oligonucleotides. The base is connected to the oxygen atom either via i) a hydrocarbon chain, which is substituted with a protected hydroxymethyl group, or via ii) a furan ring or pyrane ring or any modified furan or pyrane ring, suitable for use in the synthesis of modified oligonucleotides.

The chelating agent can be introduced into oligonucleotides with the aid of an oligonucleotide synthesizer. A useful method is disclosed in U.S. Pat. No. 6,949,639 and EP1308452. These patent publications disclose a method for direct attachment of a desired number of conjugate groups to the oligonucleotide structure during chain assembly. The chelating agents are introduced during the chain assembly. Conversion to the lanthanide chelate takes place after the synthesis during or after the deprotection steps. The carboxylic acid protecting group R″ is preferable a group that can be removed by treatment with base, such as hydroxide ion, ammonia and amine. Suitable protecting groups are methyl and ethyl groups.

According to one embodiment Z² is a radical of any of the bases thymine, uracil, adenine, guanine or cytosine, and the base is connected to the oxygen atom via i) a hydrocarbon chain, which is substituted with a protected hydroxymethyl group, or via ii) a furan ring having a protected hydroxymethyl group in its 4-position and optionally a hydroxyl, protected hydroxyl or modified hydroxyl group in its 2-position.

According to one embodiment the reactive group—Z¹—O—P(NR⁴R⁵)—O—R³ is selected from the group consisting of:

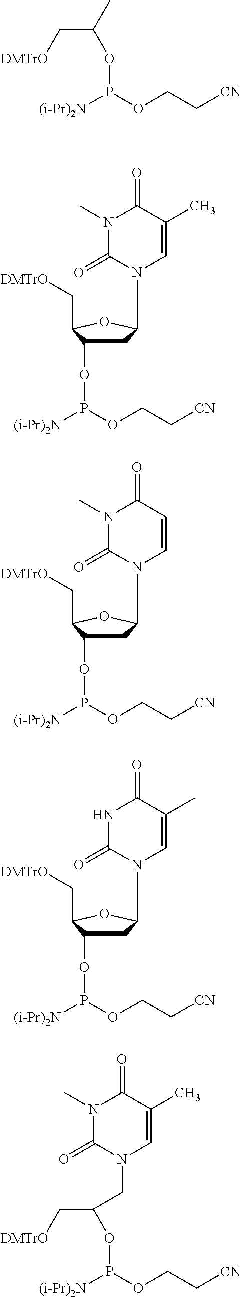

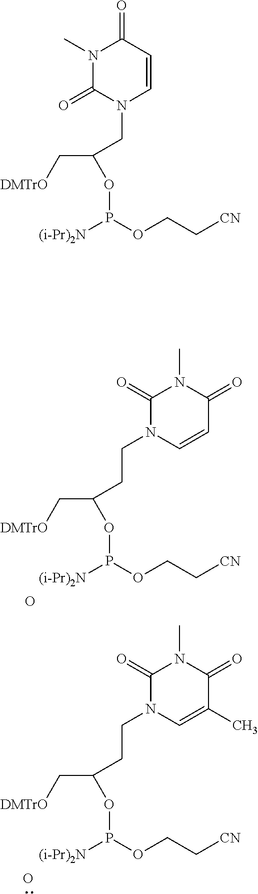

wherein—is the position of the linker L and DMTr is dimethoxytrityl.

For the preparation of oligonucleotide conjugates tethered to a single label molecule Z² can be omitted from the structure. Exemplary chelating agents suitable for oligonucleotide synthesis have the following structures:

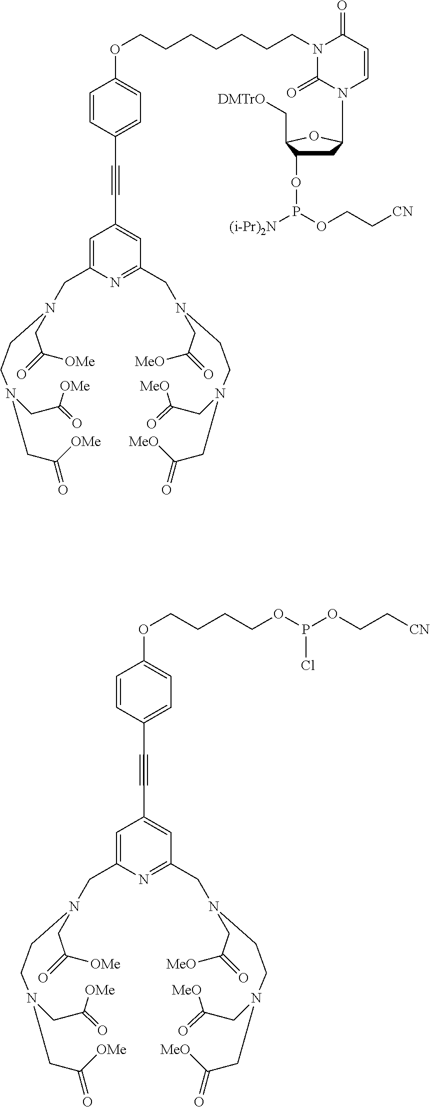

The biomolecule conjugated with a chelating agent or a chelate according to this invention is an oligopeptide, oligonucleotide, DNA, RNA, modified oligo- or polynucleotide, such as phosphoromonothioate, phosphorodithioate, phosphoroamidate and/or sugar- or base modified oligo- or polynucleotide, protein, oligosaccaride, polysaccaride, phospholipide, PNA, LNA, antibody, steroid, hapten, drug, receptor binding ligand and lectine.

According to another embodiment the present technology concerns a method of carrying out a specific bioaffinity assay using a biomolecule conjugated with a chelate of the present technology with an analyte to be determined.

According to another embodiment the present technology concerns use of a biomolecules conjugated with the chelates of the present technology in a specific bioffinity binding assay utilizing fluorometric or time-resolved fluorometric determination of a specific luminescence. The specific bioffinity assay is preferably selected from selected from a heterogenous immunoassay, a homogenous immunoassay, a DNA hydridization assay, a receptor binding assay, an immunological assay and an immunohistochemical assay.

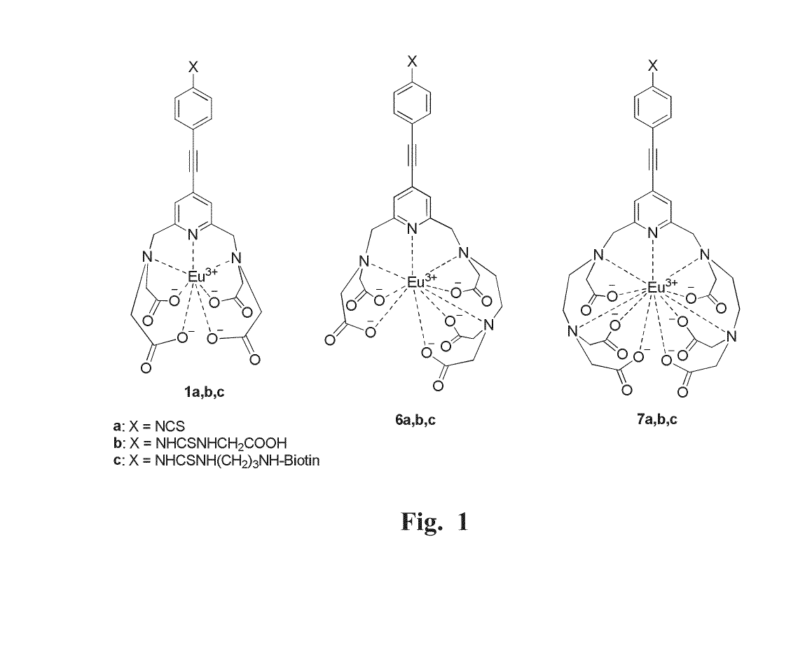

The essential difference in the structure of the chelates prior art including a single pyridine subunit and the chelates of the present technology can be seen in FIG. 1. Although there is no desire to be related to any theory, it is thought that the presence of additional carboxylic acid chelating groups enhance the stability and quantum yield compared to the chelates of the art comprising similar chromophore.

The invention will be illuminated by the following non-restrictive examples.

### EXAMPLES

The synthetic routes employed in the experimental part are depicted in Scheme 1. Formulas of compounds 6a-c and 7a-c are shown in FIG. 1.

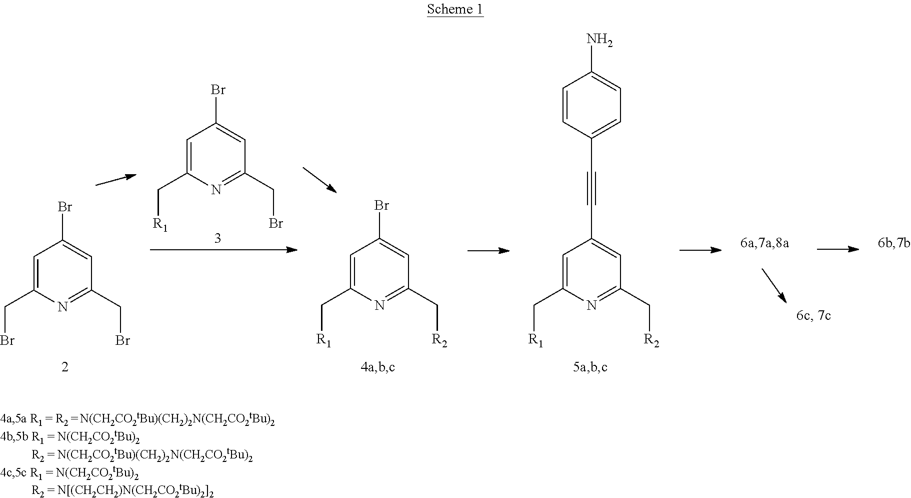

### Experimental Procedures

All reagents and solvents used were of reagent grade. The ¹H and ¹³C NMR spectra were recorded on a Bruker Avance 600 MHz NMR spectrometer operating at 600.1337 MHz and 150.9179 MHz for ¹H and ¹³C, respectively. HR Mass spectra were recorded on a Bruker micrOTOF-Q mass spectrometer. UV-visible absorption spectra were recorded on a Specord 205 (Analytik Jena) spectrometer. Steady state emission and excitation spectra were recorded on a Horiba Jobin Yvon Fluorolog 3 spectrometer working with a continuous 450 W Xe lamp. Detection was performed with a Hamamatsu R928 photomultiplier. All spectra were corrected for the instrumental functions. When necessary, a 399 nm cut-off filter was used to eliminate the second order artifacts. Phosphorescence lifetimes were measured on the same instrument working in the phosphorescence mode, with 50 μs delay time and a 100 ms integration window.

Emission decay profiles were fitted to mono-exponential and bi-exponential function using the FAST program from Edinburgh Instrument or with the Datastation software from Jobin Yvon. Hydrations numbers, q, were obtained using equation (1), were τ_{H2O} and τ_{D2O} respectively refer to the measured luminescence decay lifetimes (in ms) in water and deuterated water, using A_{Eu}=1.2 and a_{Eu}=0.25 for Eu^{III}:

q=A_{Ln}(1/τ_{H}_{₂}_{O}−1/τ_{D}_{₂}_{O}−a_{Ln}) (1)

Luminescence quantum yields were measured according to conventional procedures, with diluted solutions (optical density <0.05), using [Ru(bipy)₃]Cl₂ in nondegassed water (Φ=4.0%) as reference. The estimated relative error is ±15%.

### Example 1

Synthesis of Tetra-tert-butyl 2,2′,2″,2′″-{{{[(4-bromopyridine-2,6-diyl)bis(methylene)]bis([2-(tert-butoxy)-2-oxoethyl]azanediyl}}bis(ethane-2,1-diyl)}bis(azanetriyl)}tetraacetate (4a).

A mixture of 4-bromo-2,6-bis(bromomethyl)pyridine (2; 344 mg, 1.0 mmol), tert-butyl{[bis(ter/-butoxycarbonyl methyl)aminoethyl]amino}tris(acetate) (843 mg, 2.10 mmol) and dry potassium carbonate (1.38 g, 10 mmol) in dry acetonitrile (50 mL) was stirred overnight at 55° C. The solid was removed by filtration. The solvent was evaporated in vacuo and the product was purified on a silica gel column using 1% (v/v) triethylamine in dichloromethane as the eluent. Yield was 0.56 g (57%). ¹H NMR (CDCl₃): δ 7.59 (s, 2H), 3.79 (br, 4H), 3.39 (s, 8H), 3.33 (br, 4H), 2.84 (br, 8H), 1.40 (s, 18H), 1.38 (s, 36H). ¹³C NMR (CDCl₃): δ 170.48, 160.79, 134.32, 124.09, 80.88, 59.79, 56.05, 53.40, 52.63, 52.06, 28.12, 28.10. HR-MS for C₄₇H₈₁BrN₅O₁₂⁺: required 986.5060 and 988.5040, found 986.4993 and 988.4985.

### Example 2

Synthesis of di-tert-butyl 2,2′-{{{6-{{{2-(bis[2-(tert-butoxy)-2-oxoethyl]amino}ethyl}[2-(tert-butoxy)-2-oxoethyl]amino}methyl}-4-bromopyridin-2-yl}methyl}azanediyl}diacetate (4b).

Di-tert-butyl 2,2′-{[4-bromo-6-(bromomethyl)pyridin-2-yl]methylenenitrilo}bis(acetate) (3) (385 mg, 0.76 mmol), tert-butyl{[bis(tert-butoxycarbonylmethyl)aminoethyl]amino}acetate (319 mg, 0.80 mmol) and potassium carbonate (dry) (524 mg, 3.80 mmol) was stirred overnight at 55° C. The solid was removed by filtration. The solvent was evaporated in vacuo and the product was purified on a silica gel column using 20% (v/v) of ethyl acetate in petroleum ether as the eluent. Yield was 562 mg (89%). ¹H NMR (CDCl₃): δ 7.69 (s, 1H), 7.59 (s, 1H), 3.94 (s, 4H), 3.41 and 3.40 (2s, 10H), 2.86 (br s, 4H), 1.41 (s, 27H), 1.38 (s, 18H). ¹³C NMR (CDCl₃): δ 170.45, 170.29, 160.60, 134.49, 124.39, 124.26, 81.10, 81.00, 59.65, 59.42, 56.01, 55.81, 52.64, 51.88, 28.13, 28.11. HR-MS for C₃₉H₆₆BrN₄O₁₀⁺: required 829.3957 and 831.3937, found 829.4050 and 831.4038.

### Example 3

Synthesis of tetra-tert-butyl 2,2′,2″,2′″-(((((4-((4-aminophenyl)ethynyl)pyridine-2,6-diyl)bis(methylene))bis((2-(tert-butoxy)-2-oxoethyl)azanediyl))bis(ethane-2,1-diyl))bis(azanetriyl))- tetraacetate (5a)

Compound 4a (364 mg, 0.37 mmol) and 4-ethynylaniline (52 mg, 0.44 mmol) in the mixture of THF (10 mL) and DIPEA (10 mL) was deaerated with nitrogen for 5 min. Pd(PPh₃)₂Cl₂ (10.4 mg, 0.015 mmol) and CuI (2.9 mg, 0.015 mmol) were added as the catalysts, and the reaction mixture was stirred overnight under nitrogen at 60° C. The solvents were removed in vacuo and the product was purified on a silica gel column using a mixture of 20-30%) (v/v) of ethyl acetate in petroleum ether containing 1% (v/v) triethylamine as the eluent. Yield was 290 mg (76%). ¹H NMR (CDCl₃, 50° C): δ 7.44 (s, 2H), 7.30 (d, J=8.45 Hz, 2H), 6.61 (d, J=8.50 Hz, 2H), 3.96 (br s, 4H), 3.43 (s, 8H), 3.40 (br s, 4H), 2.90 (b, 8H), 1.46 (s, 18H), 1.42 (s, 36H). ¹³C NMR (CDCl₃, 50° C): δ 170.55, 158.78, 147.56, 133.32, 132.57, 122.66, 114.54, 111.47, 85.87, 80.83, 80.75, 60.07, 56.24, 52.88, 52.47, 28.17, 28.13. HR-MS for C₅₅H₈₇N₆O₁₂⁺: required 1023.6376, found 1023.6308.

### Example 4

Synthesis of di-tert-butyl 2,2′-(((4-((4-aminophenyl)ethynyl)-6-(((2-(bis(2-(tert-butoxy)-2-oxoethyl)amino)-ethyl)(2-(tert-butoxy)-2-oxoethyl)amino)methyl)pyridin-2- yl)methyl)azanediyl)diacetate (5b)

The synthesis was performed as above for compound 5a but using 4b (562 mg, 0.68 mmol) as the starting material. Yield was 421 mg (72%). ¹H NMR (CDCl₃): δ 7.45 (s, 1H), 7.35 (s, 1H), 7.18 (d, J=8.46 Hz, 2H), 6.51 (d, J=8.52 Hz, 2H), 4.17 (br s, 2H), 3.91 (s, 2H), 3.81 (s, 2H), 3.38 (s, 4H), 3.35 (s, 4H), 3.27 (s, 2H), 2.77 (t, d, J=6.54, 25.21 Hz, 4H), 1.37 (s, 27H), 1.33 (s, 18H). ¹³ C NMR (CDCl₃): δ 170.68, 170.55, 170.38, 159.06, 158.64, 147.93, 133.18, 133.15, 122.47, 122.38, 114.38, 110.70, 94.80, 85.66, 80.89, 80.72, 80.70, 60.21, 60.08, 59.63, 56.14, 56.09, 55.71, 52.55, 52.16, 28.09, 28.07, 28.04. HR-MS for C₄₇H₇₂N₅O₁₀⁺: required 866.5274, found 866.5294.

### Example 5

Preparation of the complex 6a.

Compound 5b (220 mg, 0.215 mmol) was dissolved in TFA (2 mL) and the mixture was stirred in a water-bath at 25° C. for 2 hours. All volatiles were removed in vacuo. The residue was dissolved in water (2 mL), a EuCl₃ solution (0.236 mmol in 0.5 mL water) was added and stirred for 10 min. The pH was adjusted to 7.0 with triethylamine and the mixture was stirred for another 10 minutes. The pH was adjusted to 9.0 with sat. Na₂CO₃. The precipitate formed was removed by centrifugation. The pH of the solution was adjusted to 7.0 with acetic acid. Acetone (45 mL) was added and the mixture was shaken for 1 min. The precipitate was collected by centrifugation, washed with acetone (50 mL) and dried with airflow. HR-MS for C₂₇H₂₇EuN₅O₁₀⁻: required 732.0961 and 734.0975, found 732.1024 and 734.1032. The precipitate was dissolved in water (1 mL). Chloroform (1 mL) and thiophosgene (0.33 mL, 4.3 mmol) were added and the mixture was stirred vigorously for 5 minutes. The pH was monitored and kept at 7.0 with 15% of NaHCO₃ solution. Chloroform was removed followed by addition of acetone (50 mL) with stirring. The precipitate formed was isolated by centrifugation, washed with acetone (50 mL) and dried. HR-MS for C₂₈H₂₅EuN₅O₁₀S⁻: required 774.0526 and 776.0539, found 774.0649 and 776.0694.

Example 6

Preparation of complex 7a

The title compound was prepared as described above for compound 6a but using compound 5a (234 mg, 0.27 mmol) as the starting material. HR-MS for the amino form C₃₁H₃₃EuN₆O₁₂²⁻: required 416.0683 and 417.0690, found 416.0721 and 417.0740. HR-MS for the isothiocyanato form C₃₂H₃₁EuN₆O₁₂S²⁻: required 437.0465 and 438.0472, found 437.0521 and 438.0512.

### Example 7

Preparation of glycine-complexes 1b, 6b and 7b.

The isothiocyanates (1a, 6a, 7a; 20 mg each) were allowed to react with glycine (300 mg) at pH ca 7. The product was purified by HPLC (column: Supelco Ascentis RP-Amide, 21.2 mm·25 cm. Particle 5 μm, flow rate 8.0 mL/min; eluent 20 mM TEAA buffer in 2-25% acetonitrile, v/v). The fractions were collected and concentrated. The salts were removed on HPLC by using the above mentioned system by omitting the buffer component from the eluent.

HR-MS for 1b C₂₆H₂₂EuN₅O₁₀S²⁻: required 373.5148 and 374.5155, found 373.5163 and 374.5173.

HR-MS for 6b C₃₀H₂₉EuN₆O₁₂S²⁻: required 424.0387 and 425.0393, found 424.0398 and 425.0380.

HR-MS for 7b C₃₄H₃₆EuN₇O₁₄S²⁻: required 474.5625 and 475.5632, found 474.5676 and 475.5690.

Example 8

Preparation of Biotin-complexes of 1c, 6c and 7c.

The isothiocyanates (1a, 6a, 7a; 1 mg each) were allowed to react with N-Biotinyl-3-aminopropylamine (TFA salt, 5 mg each) at pH ca 8.0. The product was purified by HPLC (column: Supelco Ascentis RP-Amide, 4.6 mm×15 cm, particle 5 μm, flow rate 1.0 mL/min; eluent 20 mM TEAA buffer at pH 7.0 with 2-60% methanol, v/v). The fractions were collected and dried in vacuum.

HR-MS for 1c C₃₇H₄₁EuN₈O₁₀S²⁻: required 486.0798 and 487.0805, found 486.0764 and 487.0780.

HR-MS for 6c C₄₁H₄₈EuN₉O₁₂S²⁻: required 536.6037 and 537.6044, found 536.6014 and 537.6023.

HR-MS for 7c C₄₅H₅₅EuN₁₀O₁₄S²⁻: required 587.1275 and 588.1282, found 587.1251 and 588.1260.

### Example 9

Preparation of the complex 8a

The isothiocyate chelate 8a is synthesized as described above for 7a i.e. starting from 3, but using tetra-tert-butyl 2,2′,2″,2″-(azanediylbis(ethane-2,1-diyl))bis(azanetriyl))tetraacetate synthesized as disclosed in Bioconjugate Chem. 2008, 19, 1505-1509.

### Example 10

Conditional stability constants determination.

In a typical experiment batches of 2 mL of ca 2×10⁻⁵ M solutions of the europium complexes in TRIS buffer at pH=7.4 were mixed with various quantities of a stock solution of 5×10⁻² M EDTA (or DOTA) in the same buffer, so that the ratio of added EDTA per Eu complex varies from 0 to 450000. For each solution, the emission spectrum was measured and the decrease in intensity was fitted to equation (2) using the non-linear regression analysis of the SPECFIT software

$$[ EuL ] + EDTA ↔ [ Eu ( EDTA ) ] + L \\ with K = \frac{[ Eu ( EDTA ) ] \times [ L ]}{[ EDTA ] \times [ EuL ]} = \frac{{K}_{condEDTA}}{{K}_{condL}} \qquad ( 2 )$$

The conditional stability constant could be determined using values of K_{condEDTA} calculated from literature data.

### Example 11

hCRP immunoassay.

Human C-reactive protein (CRP) was obtained from (Orion Diagnostica, Espoo, Finland) and monoclonal anti-CRP antibody 6404 was from Medix Biochemica (Kauniainen, Finland). Nunc C12 low fluor maxi wells (Thermo Scientific, Roskilde, Denmark) were coated with 150 ng of 6404 antibody in 40 μl 50 mM phosphate buffer pH 7.4 for 16 h at 4° C. The wells were washed twice using wash buffer from Kaivogen Oy (Turku, Finland). Final blocking of the well surface was carried out with 200 μl 0.1% BSA in the phophate buffer for 2 hours at 25° C. The same 6404 antibody was conjugated with d-biotin NHS ester (Sigma-Aldrich, St. Louis, USA) using 20-fold excess in 50 mM phosphate buffer pH 7.8. After conjugation the antibody was purified with NAP-5 column (GE Healthcare, Uppsala, Sweden) using TBS-buffer (50 mM Tris-Cl pH 7.5, 150 mM NaCl). Three replicates of 0-10 mg/L hCRP were incubated for 60 min in 50 μl of TBS-buffer in prewashed 6404 coated wells. The wells were washed once, and 50 ng of biotinylated 6404 antibody was added to the wells in 50 μl of TBS and incubated for 60 min. The wells were washed once and 50 ng streptavidin (BioSpa, Milan, Italy) in 50 μl of TBS was added, incubated for 10 minutes and washed once. Thereafter, 50 μl of 40 nM biotinylated europium chelates were added and incubated for 10 minutes. The wells were washed twice and measured with a Victor² 1420 multilabel counter (PerkinElmer, Wallac OY, Turku, Finland) in time-resolved luminescence mode using an excitation wavelength of 340 nm and an emission wavelength of 615 nm, a 400 μs delay, and 400 μs integration times.

Spectroscopic properties of the chelates. The spectroscopic properties of the complexes were measured in 0.01 M TPJS/HCl buffer at pH 7.4 on the glycine functionalized complexes (1b, 6b, 7b), and the most important parameters are gathered in Table 1.

TABLE 1
Spectroscopic data for the Eu complexes in 0.01M TRIS/HCl buffer at pH 7.4

| Compound | λₘₐₓ/nm | ε/M⁻¹ · cm⁻¹ | τ_{H2O}/ms | τ_{D2O}/ms | q | φ_{Ov} | η_{eff} | Φ_{Eu} |
|---|---|---|---|---|---|---|---|---|
| 1b | 319 | 16350 | 0.39 | 2.10 | 2.2 | 0.06 | 0.54 | 0.11 |
| 6b | 317 | 24350 | 1.15 | 1.96 | 0.1 | 0.14 | 0.45 | 0.31 |
| 7b | 318 | 20900 | 1.07 | 1.69 | 0.1 | 0.12 | 0.41 | 0.29 |

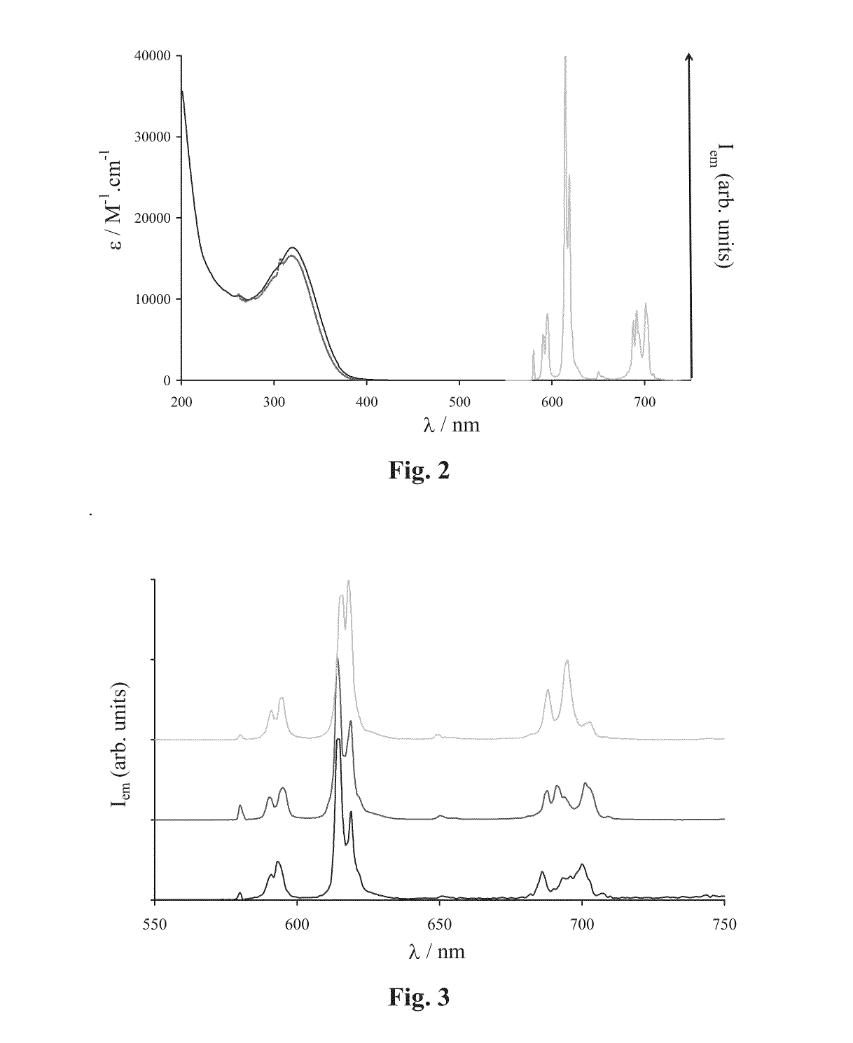

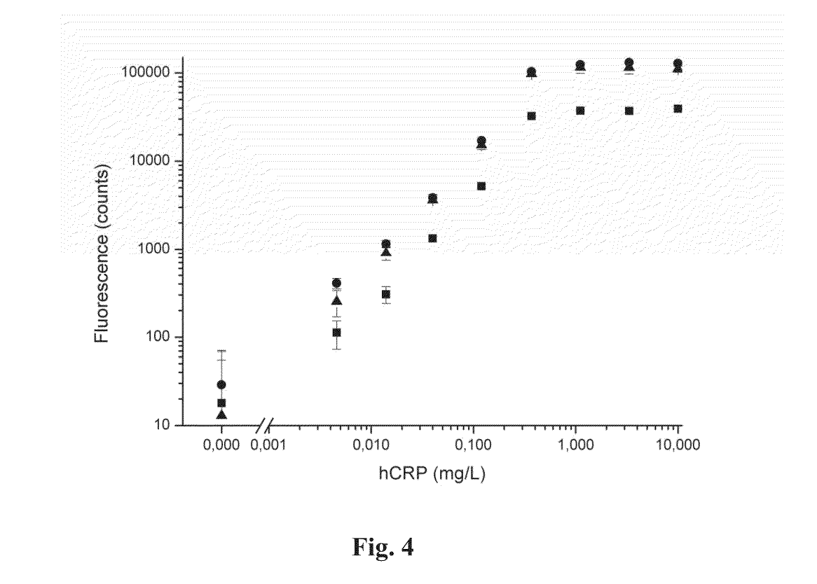

The UV-Vis absorption spectra of the three Eu complexes are very similar, displaying a strong absorption band centred at ca 318 nm, corresponding to π→π* transitions on the pyridyl rings (see FIG. 2 for 1b). The presence of the para-(thiourea)-toluyl substitution resulted in a strong bathochromic shift of this absorption band, when compared to non substituted pyridines for which the maximum of absorption can be found at 265-267 nm. Upon excitation into the π→π* absorption bands, all complexes display well resolved emission bands between 575 and 730 nm associated to f-f transitions on the europium atom. These emission bands correspond to the ⁵D₀→⁷F_{J} transitions with J=0 (single band at 575 nm), J=1 (between 578 and 600 nm), J=2 (strong, 605 to 625 nm), J=3 (weak around 650 nm) and J=4 (690 to 715 nm). The corresponding excitation spectra are perfectly superimposable with the absorption spectra, evidencing an efficient ligand to metal energy transfer process that is a good antenna effect. The luminescence decay profiles measured at the maximum of emission were all perfectly fitted with mono-exponential functions, pointing to the presence of single species in solution. The complex obtained from the heptadentate ligand of 1b displayed the shorter lifetime (0.39 ms), the coordination sphere of the europium being probably unsaturated. This was confirmed by the calculation of the hydration numbers of the complexes according to the method developed by Horrocks using Beeby's coefficients (Table 1). While the heptadentate ligand of lb releases the place for two inner sphere water molecules, the replacement of one or two acetate functions by iminodiacetate ones resulted in the fulfilment of the coordination sphere and the removal of solvent molecules from the first coordination sphere.

The Eu centred emission spectra of the complexes are presented in FIG. 3. The ⁵D₀→⁷F₂ transition represents the most intense emission band. The main variations in the series are observed on this transition and on the pattern of the ⁵D₀→⁷F₄ transition, this transition becoming more intense (compared those with J=0 to 3) for 7b.

Based on the emission spectra of the three complexes (FIG. ₂), it was possible to determine the europium centred quantum yield, Φ_{Eu} from which one can calculate the sensitization efficiency, η_{eff}, that reflects the capacity of the ligand to transfer the absorb energy to the europium, using equation

Φ_{Ov}=η_{eff}×Φ_{Eu}

Where Φ_{Ov} is the overall luminescence quantum yield measured by direct method (Table 1).

The calculated values of η_{eff} and Φ_{Eu} evidenced that the sensitization is almost the same for all compounds ranging from 41 to 54%, as expected for a similar antenna unit. In contrast, the metal centred quantum yield of 1b is largely affected by the presence of the two inner sphere water molecules, losing two third of the value obtained when the coordination sphere is saturated.

Determination of the conditional stability constants. The determination of the conditional stability constants of the different complexes was addressed by means of competition experiments with EDTA and DOTA. Only in the case of lb it was possible to displace the equilibrium in the presence of EDTA. Considering a conditional stability constant of 14.53 Log units for EDTA at this pH, the fitting resulted in a conditional stability constant of 16.7 Log units for 1b at pH=7.4. For both 6b and 7b, even in the presence of large excess of EDTA, it was not possible to displace the equilibrium, and to extract the Eu atom from its coordinating ligand. Even DOTA which is known to form very stable complexes with Ln cations was not able to demetallate the chelates of the present technology even after three days at 80° C.

Conjugation to Bioactive Molecules and hCRP immunoassay. The applicability of the chelates was demonstrated in a sandwich-type hCRP immunoassay. Therefore, the chelates 1b, 6b and 7b were conjugated with N-Biotinyl-3-aminopropylamine. The synthesized chelates 6c and 7c performed equally well in the hCRP assay. The biotin-chelate 1c resulted in lower luminescence signal than the 6c and 7c which is in accordance with the measured quantum yields and emission lifetimes. The analytical detection limits for 1c, 6c and 7c were 6.5, 1.5 and 1.9 μg/L respectively as calculated from 3 SD above the mean of the zero hCRP concentration and from the equations 1c: y=32515x−34, R²=0.96; 6c: y=87730x+16, R²=0.99; 7c: y=90760x−31, R²=0.99.

It will be apparent for an expert skilled in the field that other embodiments exist and do not depart from the spirit of the invention. Thus, the described embodiments are illustrative and should not be construed as restrictive.

1. A chelate comprising a lanthanide(III) ion selected from europium, terbium, samarium and dysprosium and a chelating ligand of formula (I) 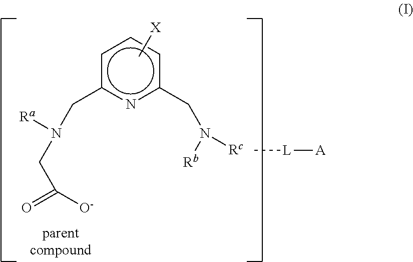
wherein
X is an aromatic unit,
L is a linker formed from one to ten moieties, each moiety being selected from the group consisting of phenylene, alkylene containing 1-12 carbon atoms, ethynydiyl (—C≡C—), ethylenediyl (—C═C—), ether (—O—), thioether (—S—), amide (—CO—NH—, —CO—NR′—, —NH—CO— and —NR′—CO—), carbonyl (—CO—), ester (—COO— and —OOC—), disulfide (—SS—), sulfonamide (—SO₂—NH—, —SO₂—NR′—), sulfone (—SO₂—), phosphate (—O—PO₂—O—), diaza (—N═N—), and tertiary amine, wherein R′ represents an alkyl group containing less than 5 carbon atoms, and L replacing a hydrogen anywhere in the parent compound, or not present,
A is a reactive group selected from isothiocyanate, bromoacetamido, iodoacetamido, maleimido, 4,6-dichloro-1,3,5-triazin-2-ylamino, pyridyldithio, thioester, aminooxy, azide, hydrazide, amino, alkyne, a methacroyl group, carboxylic acid, acid halide, and an active ester, 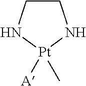
wherein A′ is cleaving group selected from Cl, (CH₃)₂SO, H₂O, and NO₃ wherein—is the position of the linker L and 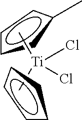
wherein—is the position of linker L,
and A replacing a hydrogen of the aromatic unit X when the linker L is not present, and
R^{a} is selected from —CH₂COO⁻ and —(CH₂)ₙN(CH₂COO⁻)₂, wherein n is 2 or 3, and
R^{b} is −(CH₂)ₘN(CH₂COO⁻)₂, wherein m is 2 or 3, and
R^{c} is selected from CH₂COO⁻, and —(CH₂)₁N(CH₂COO⁻)₂, wherein 1 is 2 or 3.

2. The chelate according to claim 1 wherein the aromatic unit is selected from phenylethynyl, furyl, thienyl, phenyl, and pyrrole, and their substituted derivatives and wherein said substituents are selected from alkyl groups and alkoxy groups.

3. The chelate according to claim 1 wherein R^{a} is selected from —CH₂COO⁻ and −CH₂CH₂N(CH₂COO⁻)₂, R^{b} is —CH₂COO⁻, and R^{c} is —(CH₂)₂N(CH₂COO⁻)₂.

4. The chelate according to claim 1 wherein the chelating ligand has the formula (III) 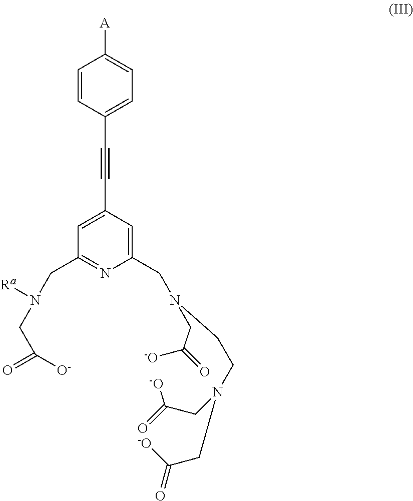
wherein the reactive group A is selected from amino, iodoacetamido, isothiocyanato and 4,6-dichloro-1,3,5-triazin-2-ylamino, and the lanthanide is selected from europium and samarium.

5. The chelate according to claim 3 wherein the lanthanide is europium.

6. The chelate according to claim 1 wherein the aromatic unit is alkoxyphenyl group and the lanthanide is selected from terbium and dysprosium.

7. The chelate according to claim 1 wherein R^{a} is −CH₂COO⁻.

8. A chelating agent of formula II 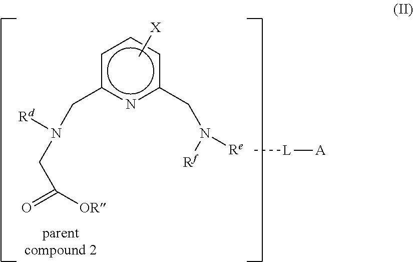
wherein X is an aromatic unit,
L is a linker formed from one to ten moieties, each moiety being selected from the group consisting of phenylene, alkylene containing 1-12 carbon atoms, ethynydiyl (—C≡C—), ethylenediyl (—C═C—), ether (—O—), thioether (—S—), amide (—CO—NH—, —CO—NR′—, —NH—CO— and —NR′—CO—), carbonyl (—CO—), ester (—COO— and —OOC—), disulfide (—SS—), sulfonamide (—SO₂—NH—, —SO₂—NR′—), sulfone (—SO₂—), phosphate (—O—PO₂—O—), diaza (—N═N—), and tertiary amine, wherein R′ represents an alkyl group containing less than 5 carbon atoms, and L replacing a hydrogen anywhere in the parent compound 2,
A is a reactive group selected from the group consisting of carboxylic acid or its salt, acid halide, carboxylic acid ester, an amino acid residue —CH(NHR¹)R²where R¹ is a transient protecting group and R² is a carboxylic acid or its salt, carboxylic acid halide or an active ester and a group of
—Z¹—O—PZ²—O—R³
wherein
one or two of the oxygen atoms optionally is replaced by sulfur,
Z² is chloro or NR⁴R⁵,
R³ is a protecting group,
R⁴ and R⁵ are alkyl groups including 1-8 carbons,
Z¹ is absent or is a radical of a purine base or a pyrimidine base wherein the base is connected to the oxygen atom via either
a) a hydrocarbon chain, which is substituted with a protected hydroxymethyl group, or
b) a furan ring or pyrane ring,
R^{d} is selected from —CH₂COOR″ and —(CH₂)ₙN(CH₂COOR″)₂, wherein n is 2 or 3,
R^{b} is —(CH₂)ₘN(CH₂COOR″)₂, wherein m is 2 or 3, and
R^{c} is selected from CH₂COOR″, and —(CH₂)₁N(CH₂COOR″)₂, wherein 1 is 2 or 3 and wherein R″ is a protecting group.

9. The chelating agent according to claim 8 wherein R^{d} is selected from —CH₂COOR″ and
—CH₂CH₂N(CH₂COOR″)₂, R^{b} is —(CH₂)₂N(CH₂COOR″)₂, and R^{c} is —CH₂COOR″.

10. (canceled)

11. A biomolecule obtained by synthesis on a solid phase by introduction of a chelating agent of formula II 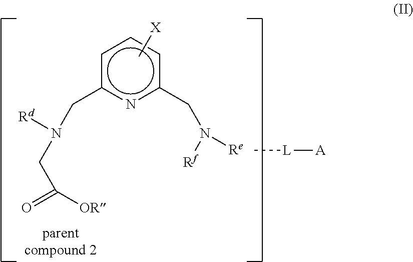
wherein X is an aromatic unit,
L is a linker formed from one to ten moieties, each moiety being selected from the group consisting of phenylene, alkylene containing 1-12 carbon atoms, ethynydiyl (—O≡C—), ethylenediyl (—C═C—), ether (—O—), thioether (—S—), amide (—CO—NH—, —CO—NR′—, —NH—CO— and —NR′—CO—), carbonyl (—CO—), ester (—COO— and —OOC—), disulfide (—SS—), sulfonamide (—SO₂—NH—, —SO₂—NR′—), sulfone (—SO₂—), phosphate (—O—PO₂—O—), diaza (—N═N—), and tertiary amine, wherein R′ represents an alkyl group containing less than 5 carbon atoms, and L replacing a hydrogen anywhere in the parent compound 2,
A is a reactive group selected from the group consisting of carboxylic acid ester, an amino acid residue —CH(NHR¹)R² where R¹ is a transient protecting group and R² is a carboxylic acid or its salt, carboxylic acid halide or an active ester and a group of
—Z¹—O—PZ²—O—R³
wherein
one or two of the oxygen atoms optionally is replaced by sulfur,
Z² is chloro or NR⁴R⁵,
R³ is a protecting group,
R⁴ and R⁵ are alkyl groups including 1-8 carbons,
Z¹ is absent or is a radical of a purine base or a pyrimidine base wherein the base is connected to the oxygen atom via either
a) a hydrocarbon chain, which is substituted with a protected hydroxymethyl group, or
b) a furan ring or pyrane ring,
R^{d} is selected from —CH₂COOR″ and —(CH₂)ₙN(CH₂COOR″)₂, wherein n is 2 or 3,
R^{b} is —(CH₂)ₘN(CH₂COOR″)₂, wherein m is 2 or 3, and
R^{c} is selected from CH₂COOR″, and —(CH₂)₁N(CH₂COOR″)₂, wherein 1 is 2 or 3 and
wherein R″ is a protecting group into the biomolecule structure followed by deprotection and introduction of a lanthanide ion.

12. The biomolecule according to claim 11 wherein said biomolecule is selected from an oligopeptide and an oligonucleotide.

13. (canceled)

14. (canceled)

15. (canceled)
